(19) United States
(12) Patent Application Publication
(10) Pub. No.: US 2004/0053295 A1
(43) Pub. Date: Mar. 18, 2004

(54) Linear array device

(76) Inventors: Alexander Wilson McKinnon, Edinburgh (GB); Changhai Wang, Edinburgh (GB)

Correspondence Address: VENABLE, BAETJER, HOWARD AND CIVILETTI, LLP, P.O. BOX 34385, WASHINGTON, DC, 20043-9998, US

(21) Appl. No.: 10/433,603

(22) PCT Filed: Dec. 5, 2001
(86) PCT No.: PCT/GB01/05389

(30) Foreign Application Priority Data
Dec. 5, 2000 (GB) 0029590.7

(51) Int. Cl. C12Q 1/68; C12M 1/34

(52) U.S. Cl. 435/6; 435/287.2

(57) ABSTRACT

The present invention provides a method of detecting the presence of at least one target analyte 14 in a fluid sample 9, as well as linear array devices 5 fo use in the method. In the method is provided a linear array device 5 comprising an elongate substrate 6 having a linear array of different spatially addressable probe moieties 7 anchored thereto. The device 5 is contacted with the sample under conditions conducive to selective binding with a probe moiety which is specific therefor. The device 5 is drawn past reading apparatus 3 for providing linear spatial address data for said probe moieties 7 and so as to detect signal indicating the presence of bound analyte 14, and the address data is correlated with bound analyte signal data so as to determine the linear spatial address of any probe moiety having analyte bound thereto, thereby to determine the identity of said probe moiety and thence indicate the presence or absence of target analyte 14.

The present invention(s) relate to devices apparatus and sensor mechanisms for the detection of target biomaterials in a fluid sample. These will generally be of use in screening and assay techniques with particular relevance to the fields of molecular biology; pharmacology and genomic or otherwise diagnostics in healthcare markets.

Physical arrays of binding agents or probes, such as oligonucleotides and polynucleotides, have become an increasingly important tool in the biotechnology industry and related fields. These arrays, in which a plurality of binding agents are deposited onto a solid support surface in the form of an array or pattern, find use in a variety of applications, including drug screening, nucleic acid sequencing, mutation analysis, and the like.

There are many different approaches to creating these so-called “genomic-chip” arrays or biological arrays and many references to methods of both attachment of biomaterials to solid surfaces in matrix arrays and their interrogation and measurement.

In terms of attachment of biomaterials to solid surface to produce such arrays for screening, U.S. Pat No. 5,412,087 describes how spatially addressable immobilisation of oligonucleotides and other Biological polymers of surfaces may be achieved—references therein discuss various methodologies that may be exploited.

The technology drivers in genomic analysis are pushing towards carrying out parallel hybridisation on array formats of biological probe-targets. In for example DNA sequencing by hybridisation; DNA fingerprinting and genetic mapping, so-called chip-array technologies are becoming the industry standard.

Technology advances are pushing better methods of attachment of materials at predefined sections of surface and for better (more specific) methods of measuring any binding to these sites during experiments.

Other than a few low-end systems that use radioactive or chemiluminescent tagging, most microarrays use fluorescent tags as their means of identification. These labels can be delivered to the DNA units in several different ways. Hence, these arrays are typically interrogated optically (e.g. U.S. Pat. No. 5,071,248), although there have been attempts to measure other physical properties, e.g.: electrical properties of these addressable sites (either conductively in liquid; U.S. Pat. No: 4,713,347, or capacitively U.S. Pat. No: 4,543,646 or as part of a field effect transistor U.S. Pat. No: 4,233,144).

The present invention provides a method of detecting the presence of at least one target analyte in a fluid sample, said method comprising the steps of:

a) providing a linear array device comprising an elongate substrate having a linear array of different spatially addressable probe moieties anchored thereto, said substrate having a leading end portion and a trailing end portion;

b) bringing said device into contact with said sample under conditions conducive to selective binding interaction between the target analyte and a said probe moiety which is specific therefor;

c) providing a microstructure reading apparatus;

d) providing relative translation of said device and said reading apparatus for providing linear spatial address data for said probe moieties anchored to said substrate;

e) reading said device so as to detect signal indicating the presence of bound analyte; and

f) correlating the address data with bound analyte signal data so as to determine the linear spatial address of any probe moiety having analyte bound thereto, thereby to determine the identity of said probe moiety.

In another aspect the present invention provides a linear array device suitable for use in a method according to the present invention, said device comprising an elongate substrate having a linear array of different spatially addressable probe moieties anchored thereto, said substrate having a leading end portion and a trailing end portion, wherein said device has at least one, longitudinally indexable, microstructure characteristic which is readable so as to provide linear spatial addresses for said probe moieties, said substrate having a tensile strength sufficient to allow stable transportation of said device through a sample contacting station and a reading station in use of said device, by means of at least one of: supporting said device with leading and trailing end portions thereof secured to spaced apart portions of a support structure, with said device extending under tension between said leading and trailing end portions, and providing relative translation between said supported device and said stations; and pulling on said leading end portion of said device.

Novel devices and methods of interrogation are provided for the diagnostic screening of liquid samples for particular biomaterials. The devices are string-like, linear arrays onto which are deposited (immobilised) spatially-addressable, “probe” biomaterials. The base material from which the string is manufactured will in general a polymer material (e.g. polyurethanes, polyesters, polycarbonates, polyureas, polyamides, polyethyleneimines, polyarylene sulfides, polysiloxanes, polyimides, polyacetates, pvdf), although these may be glass, metal, or ceramic or combinations thereof.

The addressability may be achieved through monitoring of the spatial change, created locally by the deposited/immobilised probe; which may in addition be considered in reference to other fiducial markings on the string; which may or may not be random surface textures. Such fiducial features may be exploited using correlation techniques to achieve absolute position measurement/indexing along said string and hence can allow interrogation of the biomaterial functionalised site at that position on the string. Such markings as included for position sensing may in addition contain encoded data appropriate for the string or measurement.

The string may be functionalised using a variety of techniques, however, to maximise sensitivity it is envisaged that coating said string cylindrically with biomaterials will exploit the larger surface area that this will afford when drawn through a liquid analyte.

In general, such biomaterial functionalised addressable linear-string-arrays may be used to exploit a wide variety of ligand-antiligand-type reactions. For example, direct binding assays may be performed to detect the affinity of various ligand type biomaterials (e.g. but not restricted to cell membrane receptors, monocolonal antibodies, hormones, drugs, oligonucleotides, peptides, enzymes, cofactors, lectins, sugars, oligosaccharides, cills, cellular membranes and organelles).

The result of such simultaneous screening of a liquid analyte for affinity binding to an addressable probe, anti-ligand site on the string may be interrogated, measured in a variety of ways. For example, the traditional assay techniques of auto-radiography, where one of the moieties is radio actively labelled. Fluorescence or optical measurement may also be used to measure binding to a site.

The string-like nature of the detection arrays and the small circumferential dimensions of strings lend themselves to particular local optical and/or electrical methods for measuring binding.

More generally, the linear-aspect of the string-array provides easy relative movement with respect to:

the analyte in screening;

the deposition station/mechanism in coating with Biomaterial and

the reader/detection mechanism.

In particular, in many cases the simplicity and symmetry of the system lend itself to either movement of the string relative to various other devices, however, as similar affect could have been achieved with the string stationary and moving the devices. Therefore, in further discussion of movement of the string, it will be implied, unless explicitly stated to the contrary, that this also includes such equivalent effects as could have been achieved by reciprocal movement of the device relative to a stationary string.

This simple linear transport scheme lends itself to reduction in complexity of all parts of such an array detection system. In particular:

achieving addressability/indexing and putting additional encoded information on the string—features are exploited that are unique to a point on the string; Such features may be artefacts from the production of the string, or they may be subsequently manufactured by (but not limited to) embossing, attaching, printing, imbedding, (plasma or wet) etching, abrading—either separately or in combination—which will all giving rise to a local surface or subsurface modification that may be random or periodic. These features are then used as a reference to a particular location (address) on the string. Moreover, various combinations of these methods of manufacture can be used to place either explicit or covert codes onto the strings, which may be used for identification and traceability (e.g. traceability backwards for quality control and history and forward traceability for use in subsequent bioinformatic analysis) and/or to give instructions to automated handling systems. Such codes can be either like “Gray codes” with a varying mark/space ratio or can be effectively simple “bar codes”.

deposition of the biomaterial “probes” onto the strings—the robes are deposited onto the strings relative to the index features mentioned previously. In one embodiment, this may be achieved using cylindrically depositing “drop on demand” mechanisms (e.g. ink-jet technologies);

the testing of a liquid analyte sample for “targets” simply by drawing the string through the sample (which will typically be a small volume <=200 μL and held in a loop by surface tension) or bending or folding said string into a small contained volume.

Surface binding of materials in the analyte may be challenged by ‘plucking’ or mechanically vibrating the string and/or the analyte—this provides enhanced specificity of binding detection. In addition, such agitation of the analyte/string ensures homogenous mixing of the analyte; This can be achieved using either some shaker mechanism (e.g. a piezoceramic—typically a bimorph driven at resonance of around a 20 kHz or an electromagnetic coil—like a loudspeaker coil driven at similar frequencies. Moreover, sound may be used to resonantly couple to the string or its holder and provided agitation thereby.

Reading/detecting binding—again the positioning infrastructure can be simple by drawing the ‘tested’ string through an interrogation volume where a local electrical an/or optical measurement is used to detect affinity binding.

With regard to the reading technology to topology of the string lends itself to exploiting electrical and/or optical measurement of affinity binding. This may make use of high ‘Q’ cavity structures through which the string is drawn. Measurement of a characteristic parameter of the cavity may then give a measure of the amount of binding at a site on the string. The measurement more generally may look at either absorption of stimulating energy; or in leakage of some near-field phenomena—typically a loss-type measurement. The use of a high ‘Q’ cavity provides additional amplification potential in the system that may enhance the detection sensitivity.

The present invention exploits a string or fibre, which may be of any material (e.g. polymer, metal, wire) or a composite thereof (e.g. plastic fibre with metal core of a textile)—generally it will be a linear cylindrical structure that will have ideally a high degree of flexibility. Onto this string, a spatial addressable array of biomaterials are deposited.

The scale of the strings will ideally be of a small diameter (typically <0.5 mm diameter) to allow small bending radii. The addressability of the string (to spatially locate the biomaterial with respect to the string itself) may exploit some fiducial marking which may be structured or merely some random surface texturing on the string with using some correlation measurement can give an absolute position marker/measurement along the string. Clearly a ‘tape’ could also be included.

Indexing/addressing of bio-arrays for DNA sequencing, genetic testing and diagnostics is necessary to ensure correct identification of bio-targets and to improve the efficiency of the assay process. For fluorescence detection of hybridisation of bio-molecules, it is desirable to use optical methods for indexing as this will reduce the complexity and the cost of the resultant sensor instrumentation. One method is to use a sacrificial fluorescent dye to produce an additional colour for indexing, i.e. this colour is different from those used for fluorescent labelling of probe and target molecules. Two techniques can be used to deposit the indexing dye molecules on a bio-string. The first technique involves covalent bonding of the dye molecules to the surface of the string through a linkage that is chemically added to the molecules which can be the same as that for probe immobilisation, the deposition can be realised using robotic spotting as for the probe molecules. In the second technique, the dye molecules are doped in a resin and the mixture is deposited along a fibre/string. A UV curable resin is preferred.

The second method is to produce optical elements along a bio-string for indexing. One element is an optical grating device which can be fabricated on the string. The diffraction of an optical beam can be detected and used to index the string. For polymer fibre, the fabrication methods described in the section on microstructure production will be preferred. For glass optical fibre, grating elements can be created by periodically modifying the refractive index of the fibre by projection of light from a UV laser through a phase mask or an interference pattern produced using a UV laser.

In both of the indexing methods for fluorescence detection, the indexing dye/optical grating need to be arranged in a particular periodic fashion or to follow a particular mathematical function to enable easy identification of probe locations on a string.

One simple and flexible approach involves attaching a fluorophore such as fluorescein or Cy3 to the oligonucleotide “probe” layer. Techniques for detecting fluorescence have become almost routine.

Various methods can be used to create microstructures on a string/fibre for indexing/addressing, namely hot embossing and laser micromachining. The embossing method is particularly useful for a polymer string/fibre. In this method, a master is created with the desired microstructures (e.g. optical grating for optical indexing) using UV lithography and electroforming, silicon micromaching, or precision engineering [1]. The microstructures on the master are then transferred onto a fibre by hot embossing. In this process, the polymer material is heated to a temperature around its glass transition temperature, the master is then pressed against the fibre and the fibre is cooled so a replica of the microstructures on the master is created on the surface of the fibre which can be used to index the fibre for bio-analysis.

Laser micromaching can also be used to fabricate microstructures for indexing by ablation of the surface of a fibre [2]. Light from a UV laser is projected to the fibre surface through a mask with a pattern of the desired microstructure. The transmitted light ablates the fibre surface to form a microstructure. The indexing microstructure can also be fabricated by scanning a focussed laser spot on the fibre surface.

Both of the above methods rely on the removal of some surface material to form the indexing microstructures. Alternatively simple indexing structures, such as polymer “bumps” can be produced on a fire/string by depositing additional material locally. For example, an UV curable resin may be deposited using techniques similar to that of an ink jet. Lamination through a mask can also be used for producing indexing microstructures for one dimensional bio-analysis.

Encoded information may take a variety of codes be that either simple colour coding of the strings to the eye; or the inclusion of encoded information in either applied structures (in the form of simple bar-codes or gray-codes) or other features. These codes may be used for whatever purposes—e.g. batch control; history, etc. The codes may be interlaced along the length of the string in a similar manner to that used on compact disks to provide enhanced data integrity—this can also be achieved using multiply redundant codes; in general a 128-bit code should be easily achievable, but clearly other codes are covered (figure needed)

Key advantages of this embodiment of a one dimensional flexible array will be that the biomaterial may be simply transported through liquid analytes, and detection/measurement systems.

This can allow for simplification of the mechanisms for fluidic handling of the liquid sample to be analysed and the phenomena and mechanism to be exploited. In its simplest form the string will be mounted taught on a rigid carrier which is then moved relative to some datum structure. Utilising close contact of the string with respect to simple kinematically constrained geometries e.g. v-groove technology in silicon processing will allow very precise +/−1 μm positioning of string relative to any reader—whilst allowing movement along the string. More complicatedly, the string may be included in a cassette and or may have a ferrite or magnetically-coated end that will allow dragging forces to be applied using an external magnetic field.

Generally, the liquid analyte will be less than 500 μL in volume and will exploit surface tension in its coating of the string. In one embodiment, the analyte will be sitting on a surface with controlled circular hydrophilic regions—the string is then pulled through the resulting meniscus.

Another embodiment makes use of a droplet held in a loop, or a droplet of analyte attached to and shaken along the string.

The measurement or detection of binding events is envisaged as being again simplified as the topology and scale of the string together with the simplicity of transport of the said string make it easy to exploit local measurements at a point in space. One preferred mechanism is to measure some electrical/optical property of the string, which may be ‘dry’ or in aqueous solution for the measurement. Having the string pass through a high ‘Q’ resonant cavity (at some suitable frequency—RF or optical or both) can allow for gain in the measurement performed, which can increase the sensitivity of detection. Also since the cylindrical cross-section of the string with material circumferentially around it, measurement is integrating through the whole volume of the material which again can lead to increased sensitivity.

The string-like nature of the arrays are also ideally suited to mechanically challenging the affinity binding reactions—by mechanically vibrating the string in solution (possibly just plucking it) additional mixing can be assured and also non-specific (—non affinity bound) materials may be challenged from attachment to a site. This can lead to an increase in specificity of the assays.

Further preferred features and advantages of the invention will appear from the following detailed description given by way of example of some preferred embodiments illustrated with reference to the accompanying drawings in which:

FIG. 1 is a schematic view of a sample testing system of the invention;

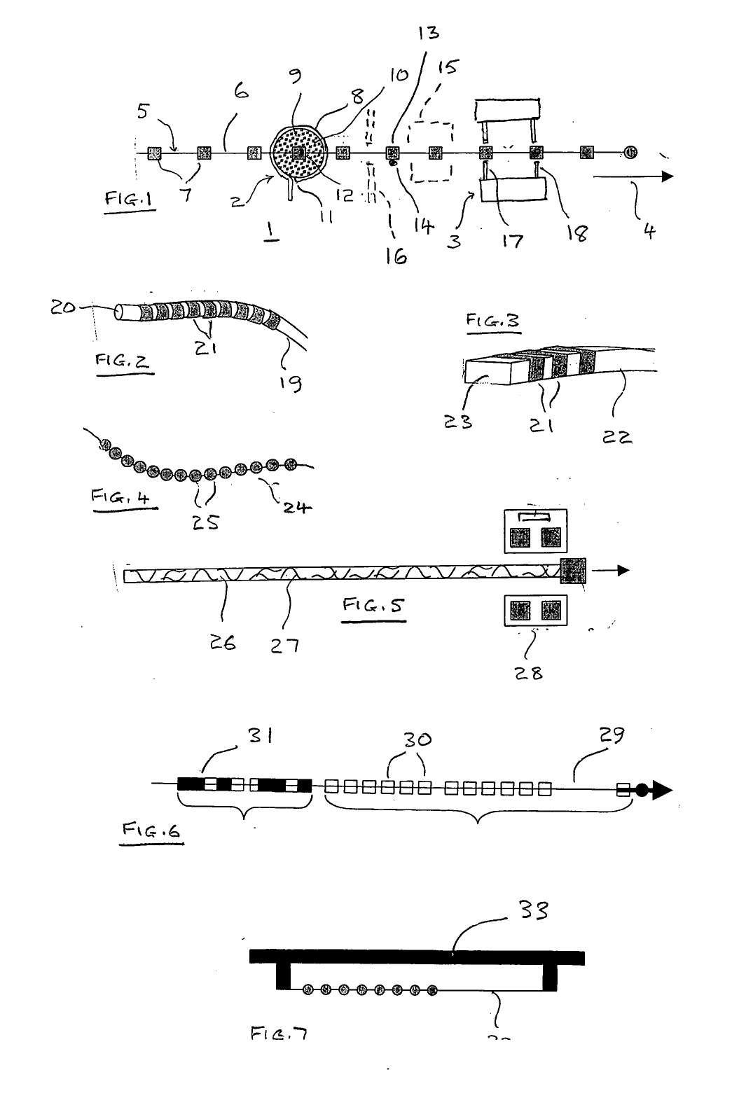

FIGS. 2 to 4 are schematic views of different linear array devices of the invention;

FIG. 5 is a schematic view showing reading of the random microstructure of an elongate substrate used in a device of the invention;

FIG. 6 is a schematic view of another linear array device of the invention;

FIG. 7 is a schematic view of a supported linear array device of the invention;

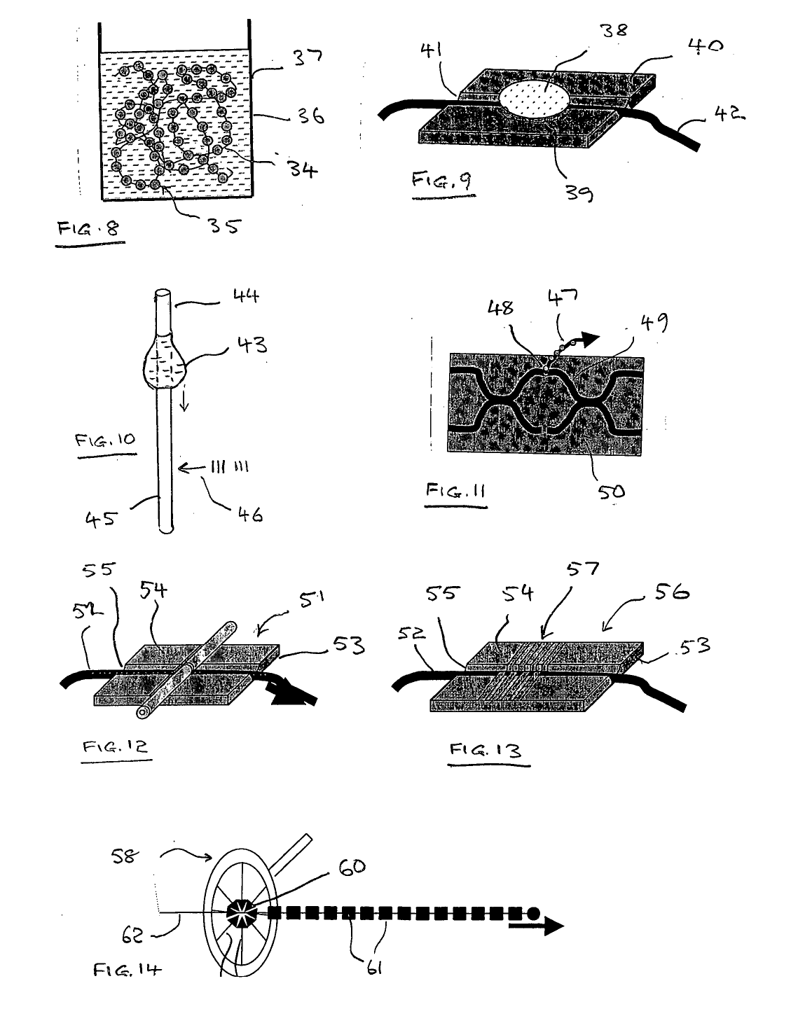

FIGS. 8 to 10 are schematic views showing alternative sample contacting arrangements;

FIGS. 11 to 13 are schematic views showing use of alternative forms of reading apparatus; and

FIG. 14 is a schematic view of an apparatus for manufacture of a linear array device of the invention by attaching probe moieties to the substrate.

FIG. 1 shows a sample testing system 1 comprising a sample contacting station 2 and a reading station 3, and a transport mechanism 4 for drawing a linear array device 5 successively past said sample contacting station 2 and said reading station 3. The linear array device 5 comprises an elongate substrate 6 having a series of different oligonucleotide probe moieties 7 anchored thereto. At the sample contacting station 2 a wire loop 8 is used to support a small volume of fluid sample 9 containing various labelled analytes 10. The loop 8 has a small opening 11 through which the linear array 5 can be passed so as to immerse part 12 of the device 5 in the sample 9. When a probe moiety 13 encounters a target analyte 14 which specifically binds thereto, this remains bound thereto when the device is withdrawn from the fluid sample 9. Normally a washing station 15 would be provided to ensure complete removal of any other parts of sample which might have become entrained with the device. Alternatively and/or additionally there could be provided a wiping or doctor device 16 for preventing entrainment of fluid from the sample contact station as the device is withdrawn from it.

The reading station 3 includes a signal reading apparatus 17 for detecting the presence of labelled target analyte 14 bound to a probe moiety 13 on the device 5, and a microstructure reading apparatus 18 for obtaining address data for said probe moiety 13 with target analyte bound thereto. This address data can then be used to identify the particular probe moiety 13 having analyte bound thereto, thereby confirming the presence of a particular target analyte 14 which hybridises with said oligonucleotide probe.

The linear array device 5 also has at its leading end portion 19 a traction engagement device in the form of a magnetic bead 20 which can be engaged by a magnetic traction device 4 to pull the linear array device 5 through the sample contacting and reading stations 2,3.

FIG. 2 shows a linear array device 19 in the form of a generally cylindrical filament 20 with a series of annular probe moiety deposits 21 extending therealong. In FIG. 3 there is shown a linear array device 22 wherein the substrate 23 is in the form of a tape. This type of substrate is particularly advantage for use with microstructure reading apparatus based on electrical capacitance measurements as this allows the separation of the capacitor plates of the reading apparatus to be minimized and the area thereof maximized. In the linear array device 24 of FIG. 4 the probe moiety deposits 25 have a generally spherical surface (by use of a beaded substrate onto which probe material is applied or by building up the amount of probe material on a regular cross-section substrate) so as to maximize the probe area presented to the sample.

FIG. 5 shows a substrate 26 which has a random microstructure characteristic 27 which is recorded during a first pass. In a subsequent pass of the linear array device containing said substrate 26, by using suitable reading apparatus 27 provided with a correlation processing device 28, unique address data can be obtained.

FIG. 6 shows schematically a linear array device 29 with a series of probe moieties 30 and with a bar coded section 31 providing identification data for the individual device 29.

FIG. 7 shows a linear array device 32 supported on a bow support structure 33 to facilitate handling etc.

FIGS. 8 to 10 are schematic views showing alternative sample contacting arrangements. In FIG. 8 the linear array device 34 is allowed to adopt a compact form 35 for complete immersion in a fluid sample 36 in a vessel 37. In FIG. 9 a fluid sample 38 is constrained in a well 39 on a plate 40 which has a groove 41 extending across it and intersecting the well 39. The groove acts as a kinematic constraint for a linear array device 42 as it is pulled along the groove 41 and through the fluid sample 38.

In FIG. 9 a drop of fluid sample 43 is applied to the leading end portion 44 of a vertically extending linear array device 45 and allowed to flow down the length of the linear array device 45. The linear array device 45 is mechanically perturbed by application of a suitable vibrational frequency from an ultrasonic sound apparatus 46 so as to encourage the fluid sample drop 43 to flow down the linear array device 45 whilst at the same time challenging to a greater or lesser degree the binding of analyte to the probe moieties.

FIGS. 11 to 13 are schematic views showing use of alternative forms of reading apparatus. FIG. 11 shows a linear array device 47 being drawn through a Fabry-Perot cavity 48 formed in one arm 49 of a Mach-Zender planar light guide interferometer 50. FIG. 12 shows a fibre optic reading apparatus 51 being used to read a linear array device 52 as it is drawn through a kinematic constraint guide support device 53 comprising a plate 54 with a groove 55 therein for receiving the linear array device 52. FIG. 13 shows schematically a reading apparatus 56 together with a kinematic constraint guide support device 53 similar to that in FIG. 12, with a reading device 57 suitable for use in changes in electrical capacitance as the linear array device 52 is drawn past it.

FIG. 14 shows schematically a linear array device production apparatus 58 based on ink-jet writing technology, which uses an annular array 59 of fluid jet ejection nozzles 60 to deposit in controlled manner an annular layer of probe material 61 onto an elongate substrate 62 as this is drawn through the array 59.

Literature References

U.S. Pat. No. 5,412,087: “Spatially-addressable immobilization of oligonucleotides and other biological polymers on surfaces”;

U.S. Pat. No. 5,482,867: “Spatially-addressable immobilization of anti-ligands on surfaces”;

U.S. Pat. No. 5,071,248: “Optical sensor for selective detection of substances and/or for the detection of

U.S. Pat. No. 4,713,347: “Measurement of ligand/anti-ligand interactions using bulk conductance”;

U.S. Pat. No. 4,233,144: “Electrode for voltammetric immunoassay”;

U.S. Pat. No. 6,210,910: “Optical fiber biosensor array comprising cell populations confined to microcavities”;

U.S. Pat. No. 5,729,009: “Method for generating quasi-sinusoidal signals”

U.S. Pat. No. 6,303,924: “Image sensing operator input device”

U.S. Pat. No. 6,231,760: “Apparatus for mixing and separation employing magnetic particles”

Barinaga, M., “Will ‘DNA Chip’ Speed Genome Initiative?” Science 253:1489 (1991).

Chetverin, A. B. and Kramer, F. R., “Oligonucleotide Arrays: New Concepts and Possibilities” Bio/Tech. 12:1093-1099 (1994).

Gerhold et al., “DNA chips: promising toys have become powerful tools” Trends in Biochemical Sciences, 24:5:168-173 (1999)

Carrano et al., “A high-resolution, fluorescence-based, semiautomated method for DNA fingerprinting” Genomics 4:129-136 (1989).

Schift et al., “Nanoreplication in polymers using hot embossing and injection moulding”, Microelectronic Engineering, Vol. 53, 171-174, (2000)

Zimmer et al., “Combination of different processing methods for the fabrication of 3D polymer structures by excimer laser machining”, Applied Surface Sciences, Vol. 154-155, 601

1. A method of detecting the presence of at least one target analyte in a fluid sample, said method comprising the steps of:
a) providing a linear array device comprising an elongate substrate having a linear array of different spatially addressable probe moieties anchored thereto, said substrate having a leading end portion and a trailing end portion;
b) bringing said device into contact with said sample under conditions conducive to selective binding interaction between the target analyte and a said probe moiety which is specific therefor;
c) providing a microstructure reading apparatus;
d) providing relative translation of said device and said reading apparatus for providing linear spatial address data for said probe moieties anchored to said substrate;
e) reading said device so as to detect signal indicating the presence of bound analyte; and
f) correlating the address data with bound analyte signal data so as to determine the linear spatial address of any probe moiety having analyte bound thereto, thereby to determine the identity of said probe moiety.

2. A method according to claim 1 wherein said device is brought into a compacted form for contacting with said sample.

3. A method according to claim 1 wherein initially the leading end portion of said device is brought into contact with said sample; and then there is provided relative translation of said device and said body of solution so that successive ones of said linear array of probe moieties are contacted with said body of solution.

4. A method according to claim 3 wherein said device is drawn through said sample.

5. A method according to claim 3 wherein said body of solution is allowed to flow along said device.

6. A method according to any one of claims 1 to 5 which includes the step of subjecting the device to contact or non-contact perturbation thereof so as to challenge the binding of any material bound to any of said probe moieties.

7. A method according to any one of claims 1 to 6 wherein said microstructure reading apparatus is used for reading microstructural changes resulting from the binding of analyte to a probe moiety to provide said analyte binding signal.

8. A method according to any one of claims 1 to 7 wherein said microstructure reading apparatus is used for reading at least one of: microstructural features of said substrate, microstructural features of said device provided by said probe moieties, and tags provided on or in said substrate, for providing address data.

9. A method according to claim 8 wherein said microstructure reading apparatus is used for a first reading of said microstructural features of said substrate prior to anchoring of said probe moieties thereto for indexing of said substrate, and said probe moieties are anchored to said substrate at predetermined linear spatial addresses based on said indexing; and said microstructure reading apparatus is used for a second reading of said device after contacting thereof with said sample.

10. A method according to claim 8 wherein said microstructure reading apparatus is used for a first reading of said device to provide linear spatial addresses for said probe moieties based on the order in which said probe moieties are anchored to said substrate along the length thereof; and said microstructure reading apparatus is used for a second reading of said device after contacting thereof with said sample for indexing of the bound analyte signal reading.

11. A method according to any one of claims 1 to 10 wherein is used a sample with labelled analyte, in which method there is provided a label reading apparatus for reading of bound analyte signal.

12. A method according to claim 11 wherein is used a label reading apparatus for reading label selected from colour, fluorescent, and radio label.

13. A method according to any one of claims 1 to 12 wherein is used a microstructure reading apparatus for reading of microstructure using at least one of electromagnetic radiation and electrical characteristics.

14. A method according to any one of claims 1 to 13 wherein is used microstructure reading apparatus for reading at least two different microstructure characteristics.

15. A method according to any one of claims 1 to 14 wherein said microstructure reading apparatus is used for reading at least one of an inherent microstructure characteristic of the substrate and an applied microstructure characteristic of the substrate.

16. A method according to any one of claims 1 to 15 which includes the preliminary step of applying at least one of a random, indexed, and encoded, microstructure characteristic to said substrate, internally and/or externally thereof.

17. A method according to claim 16 which method includes an correlation process step when reading a random microstructure characteristic for determining address locations for probe moieties having analyte bound thereto.

18. A method according to any one of claims 1 to 16 wherein is used a microstructure characteristic reading apparatus having a high Q cavity through which said device is passed.

19. A method according to any one of claims 1 to 18 wherein said device is transported past the or each said reading apparatus so that successive portions of said linear probe array are brought into reading alignment with said reading apparatus.

20. A method according to claim 19 wherein traction is applied to said leading end of said device to draw it past the or each said reading apparatus.

21. A method according to claim 20 wherein the or each said reading apparatus is provided with a kinematic constraint device for stabilizing the device against displacement orthogonal to the transport direction as it is drawn past said reading device.

22. A method according to claim 20 or claim 21 wherein said leading end of said device is provided with a traction engagement moiety.

23. A method according to claim 22 wherein is provided a magnetic traction engagement moiety and said device is transported by bringing a magnet device into proximity with said magnetic traction engagement moiety and moving said magent device so as to draw said device past the or each said reading apparatus.

24. A method according to any one of claims 1 to 18 wherein said device is supported in an extended configuration and the or each said reading apparatus is transported past said device.

25. A method according to any one of claims 1 to 24 wherein is used at least one of near-field and far-field reading apparatus.

26. A method according to any one of claims 1 to 25 wherein said device is transported successively through a sample contacting station, and a reading station in a single pass.

27. A method according to claim 26 wherein the device is transported through a washing station between said a sample contacting station and said reading station.

28. A method according to any one of claims 1 to 27 which includes the step of quantitative determination of bound analyte signal.

29. A linear array device suitable for use in a method according to claim 1, said device comprising an elongate substrate having a linear array of different spatially addressable probe moieties anchored thereto, said substrate having a leading end portion and a trailing end portion, wherein said device has at least one, longitudinally indexable, microstructure characteristic which is readable so as to provide linear spatial addresses for said probe moieties, said substrate having a tensile strength sufficient to allow stable transportation of said device through a sample contacting station and a reading station in use of said device, by means of at least one of: supporting said device with leading and trailing end portions thereof secured to spaced apart portions of a support structure, with said device extending under tension between said leading and trailing end portions, and providing relative translation between said supported device and said stations; and pulling on said leading end portion of said device.

30. A device according to claim 29 wherein said microstructure characteristic is one readable by means of at least one of electromagnetic radiation and electrical property measurement.

31. A device according to claim 29 or claim 30 wherein said microstructure characteristic has a spatial address resolution capability of not less than 500 μm.

32. A device according to claim 31 wherein said microstructure characteristic has a spatial address resolution capability of from 10 to 300 μm.

33. A device according to any one of claims 29 to 32 wherein said substrate is of at least one material selected from a natural or synthetic polymer, a metal, a ceramic, and a glass.

34. A device according to any one of claims 29 to 33 wherein said substrate has a diameter of not more than 1 mm.

35. A device according to claim 34 wherein said substrate has a diameter of from 50 to 500 μm.

36. A device according to any one of claims 29 to 35 wherein said substrate has a length of from 5 to 50 mm.

37. A device according to any one of claims 29 to 36 wherein said probe moieties are anchored to said substrate at annularly extending zones.

38. A device according to any one of claims 29 to 37 wherein said substrate has from 10 to 10,000 different probe moieties are anchored to said substrate.

39. A device according to any one of claims 29 to 38 wherein said probe moieties are anchored to said substrate by means of a covalent bond.

40. A device according to any one of claims 29 to 39 wherein said probe moieties are selected from polynucleotides, peptides, cell membrane receptors, polyclonal or monocolonal antibodies, hormones, drugs, oligonucleotides, peptides, enzymes, cofactors, lectins, sugars, oligosaccharides, cills, cellular membranes and organelles.

41. A method of making a linear array device according to claim 25 which comprises the steps of:
a) providing an elongate substrate according to claim 25; and
b) anchoring thereto different probe moieties in a linear array extending between the leading and trailing end portions thereof.

42. A method according to claim 41 wherein said probe moieties are applied to said substrate by means of a writing apparatus with a contact or non-contact fluid ejector.
